(19) United States
(12) Patent Application Publication
(10) Pub. No.: US 2011/0184429 A1
(43) Pub. Date: Jul. 28, 2011

(54) SURGICAL DEVICE

(76) Inventor: Pierre F. Saldinger, Weston, CT (US)

(21) Appl. No.: 13/013,889

(22) Filed: Jan. 26, 2011

Related U.S. Application Data
(60) Provisional application No. 61/298,586, filed on Jan. 27, 2010.

(51) Int. Cl. A61B 17/26

(52) U.S. Cl. 606/113; 606/114

(57) ABSTRACT

A surgical device for extracting material located internally within a patient including an actuator slidably positioned in a handle and movable relative thereto between a first position and a second position. A first flexible conduit having a first end coupled to the handle and defining a bore extending there through is coupled to the actuator. An actuation line having a first end coupled to the actuator and extending through the bore defined by the first flexible conduit is further included. A surgical instrument is coupled to a second end of the actuation line, upon movement of the actuator from the first position to the second position the surgical instrument moves from a retracted position within the bore defined by the first flexible conduit to an extended position wherein the surgical instrument extends at least partially from a second end of the first flexible conduit.

### CROSS-REFERENCE TO RELATED APPLICATIONS

The present application claims the benefit under 35 U.S.C. §119 (e) of the Provisional Patent Application Ser. No. 61/298,586 filed Jan. 27, 2010, the disclosure of which is incorporated herein by reference in its entirety.

### FIELD OF THE INVENTION

The disclosure relates to surgical devices and methods of utilizing the same. More particularly, the disclosure relates to a surgical device for the removal of material located internally within a patient.

### BACKGROUND OF THE INVENTION

To minimize patient pain and recovery time, surgeons often employ minimally invasive surgical techniques to perform procedures such as, but not limited to, colectomy (removal of colon), cholecystectomy (removal of the gall bladder), nephrectomy (removal of kidney), removal of polyps, lumps and/or tumors, as well as suturing and biopsy procedures. Such procedures are often referred to as laparoscopic surgeries.

Laparoscopic surgery refers to a surgical procedure that generally involves thin instruments, a laparoscope (a video camera), and several relatively small incisions made to the patient. While performing the laparoscopic procedure, the surgeon watches a video screen that displays the images provided by the video camera. The surgeon manipulates the small instruments placed in the incisions based on the images provided by the video camera.

Despite advances made in minimally invasive surgeries, not every patient is a candidate due to certain anatomical anomalies. Moreover, many procedures performed laparoscopically result in as much patient pain, hospitalization or complications as compared to a traditional “open” surgeries. This may be due to larger incisions required to remove the tissue or organ, the location of the incisions, and/or the need for additional incisions to perform the procedure.

Accordingly, there is a need for a medical device and method that allows a surgeon to perform minimally invasive surgeries while reducing or alleviating patient pain as a result of the surgery, reduced complications, reduced patient hospitalization, and reduced scarring.

### SUMMARY OF THE INVENTION

One embodiment of the present invention disclosed herein relates to a surgical device for extracting material located internally within a patient. The surgical device includes a handle, an actuator slidably positioned in the handle and movable relative thereto between a first position and a second position, a first flexible conduit having a first end coupled to the handle and defining a bore extending therethrough. The device further includes a flexible actuation line having a first end coupled to the actuator and extending through the bore defined by the first flexible conduit. The device further includes a surgical instrument coupled to a second end of the actuation line, upon movement of the actuator from the first position to the second position the surgical instrument moves from a retracted position within the bore defined by the first flexible conduit to an extended position wherein the surgical instrument extends at least partially from a second end of the first flexible conduit, wherein the surgical instrument is actuated to grippingly retain material located internally within a patient upon movement of the actuator from the second position to the first position.

Another embodiment disclosed herein relates to a method for extracting material located internally within a patient. The method comprises the step of inserting a surgical device as described above into an esophagus of a patient, wherein the surgical device extends through the esophagus to a stomach having an incision therein; passing the surgical device through the incision to an internal cavity of the patient such that the surgical device is proximate to material in need of extraction, wherein at least a portion of the material is surrounded by an extraction pouch; moving the actuator of the surgical device to a second position along the length of the handle thereby extending the at least a portion of the surgical instrument from the end of the first flexible conduit; moving the actuator to a first position along the length of the handle, thereby actuating the surgical instrument to grippingly retain at least a portion of the extraction pouch; and withdrawing the surgical device from the patient, thereby removing the extraction pouch and material from the patient.

### BRIEF DESCRIPTION OF THE DRAWINGS

FIG. 1 illustrates a surgical device in accordance with one embodiment of the present invention.

FIG. 2A illustrates a surgical device in accordance with one embodiment of the present invention.

FIG. 2B illustrates the surgical device shown in FIG. 2B.

FIG. 3 illustrates a surgical device in accordance with one embodiment of the present invention wherein the surgical device is used to extract a material from an internal location of a patient.

### DETAILED DESCRIPTION OF THE INVENTION

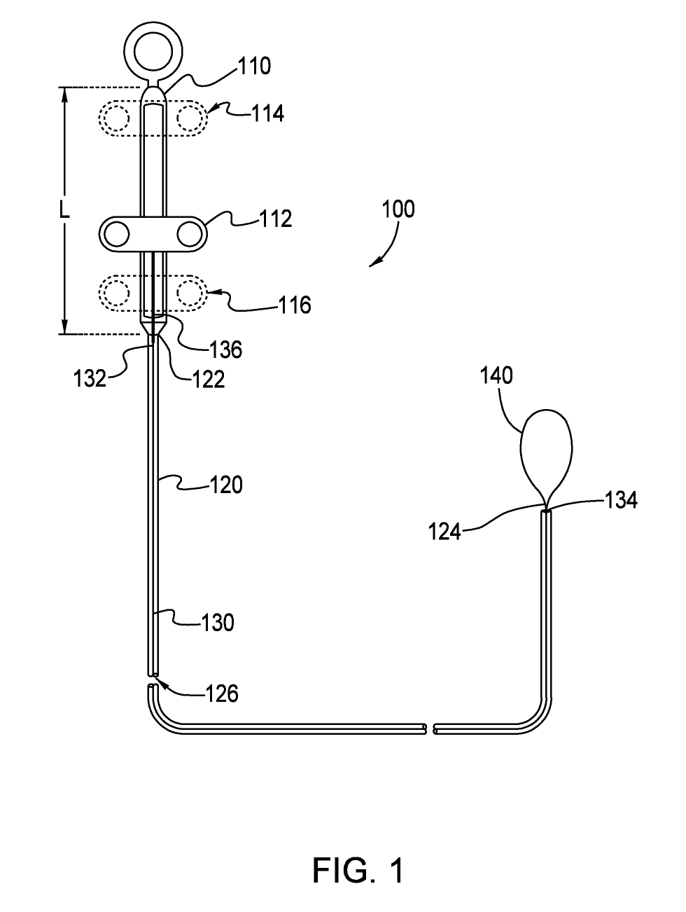

The present invention generally relates to a surgical device for use in minimally invasive surgical procedures, such as laparoscopic surgeries. In one embodiment, as shown in FIG. 1, the present invention includes a surgical device 100.

The surgical device 100 may be utilized to extract material located internally within a patient. The surgical device 100 is not limited in the material it can extract from an internal location of a patient's body. For example, the surgical device 100 may be utilized to extract an organ, such as a gall bladder or kidney, from a patient. In another example, the surgical device may be utilized to extract tissue, such as a polyp or a tumor, from a patient. In yet a further example, the surgical device 100 may be implemented to perform a biopsy of tissue within a patient, or may be implemented to remove a foreign object, such as a bullet, from a patient.

The surgical device 100 includes a handle 110 and an actuator 112 that is slidably positioned in the handle. In the illustrated embodiment, the actuator 112 is movable relative to the handle 110 between a first position and a second position as indicated by the arrow “L.”

The surgical device 100 also includes a first flexible conduit 120. The first flexible conduit 120 may be any type of conduit adaptable for surgical procedures. One example of the first flexible conduit 120 includes, but is not limited to flexible surgical tubing. While not shown in FIG. 1, it is contemplated that in one embodiment, the first flexible conduit 120 may be a multi-lumen surgical tubing, i.e., the first flexible conduit may have more than one opening with corresponding bores extending therethrough. Multiple lumens allow other devices, such as valves, to be coupled to the first flexible conduit 120.

In one embodiment, the first flexible conduit 120 is made of thermoplastic material. However, the surgical device 100 is not limited in this regard as the first flexible conduit 120 may be made of latex, silicone, or other materials.

As shown in FIG. 1, the first flexible conduit 120 has a first end 122 and an opposing second end 124. The first end 122 of the first flexible conduit 120 is coupled to the handle 110. The first end 122 may be coupled to the handle 110 in any manner, including, but not limited to, a screw portion fitted to the first flexible conduit 120 and a thread portion on the handle 110, an adhesive such as glue, a locking mechanism, and the like.

The first flexible conduit 120 defines a bore 126 that extends through the first flexible conduit. A flexible actuation line 130 is disposed in and extends through the bore 126 defined by the first flexible conduit 120. In one embodiment the flexible actuation line 130 is made from a flexible metal material. However, the surgical device 100 and the flexible actuation line 130 may be made of other materials, including, but not limited to plastic.

The flexible actuation line 130 has a first end 132 and an opposing second end 134. The first end 132 of the flexible actuation line 130 is coupled to the actuator 112. In one embodiment, as shown in FIG. 1, the first end 132 of the flexible actuation line 130 is coupled to the actuator 112 by a connecting device 136 extending from the actuator and into the bore 126. The connecting device 136 may be, for example, a rigid metal rod or a rigid plastic rod. Alternatively, the first end 132 of the flexible actuation line 130 may be coupled directly to the actuator 112 without the use of a connecting device 136. The first end 132 of the flexible actuation line 130 may be coupled to the actuator 112 in any manner acceptable, including, but not limited to an adhesive material, a screw and thread mechanism, and the like.

The second end 134 of the actuation line 130 is coupled to a surgical instrument 140. The surgical instrument 140 coupled to the actuation line 130 may be clamps, pincers, a resection device such as a blade or scissors, tweezers, articulating arms, or a combination thereof. As shown in FIG. 1, the surgical instrument 140 may be a resection loop, which is often referred to as a “snare.” The resection loop, or snare, can be formed from the actuation line.

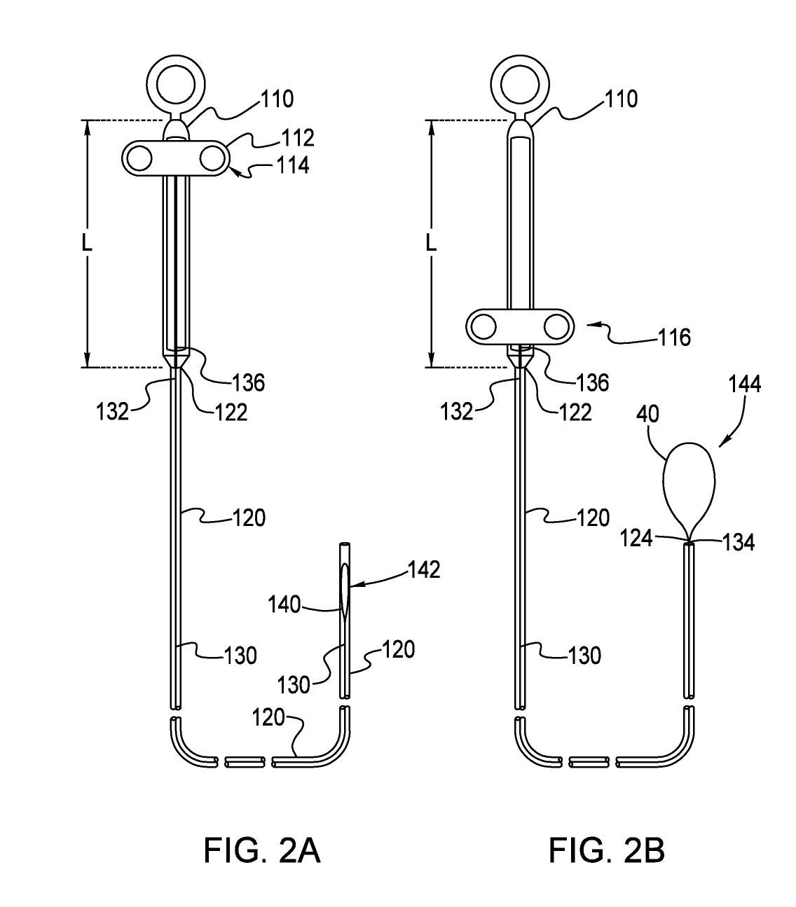

Movement of the surgical instrument 140 is caused by moving the actuator 112 and the flexible actuation line 130 coupled thereto. As shown in FIGS. 2A and 2B, in one embodiment, upon movement of the actuator 112 from the first position 114 (FIG. 2A) to the second position 116 (FIG. 2B) along the length L of the handle 110, the surgical instrument 140 moves from a retracted position 142 (FIG. 2A) within the bore 126 defined by the first flexible conduit 120 to an extended position 144 (FIG. 2B) wherein the surgical instrument 140 extends at least partially from the second end 124 of the first flexible conduit 120. When the surgical instrument 140 is actuated by movement of the actuator 11 from the first position 114 (FIG. 2A) to the second position 116 (FIG. 2B) along the length L of the handle 110 the surgical instrument 140 grippingly retains material located internally within a patient.

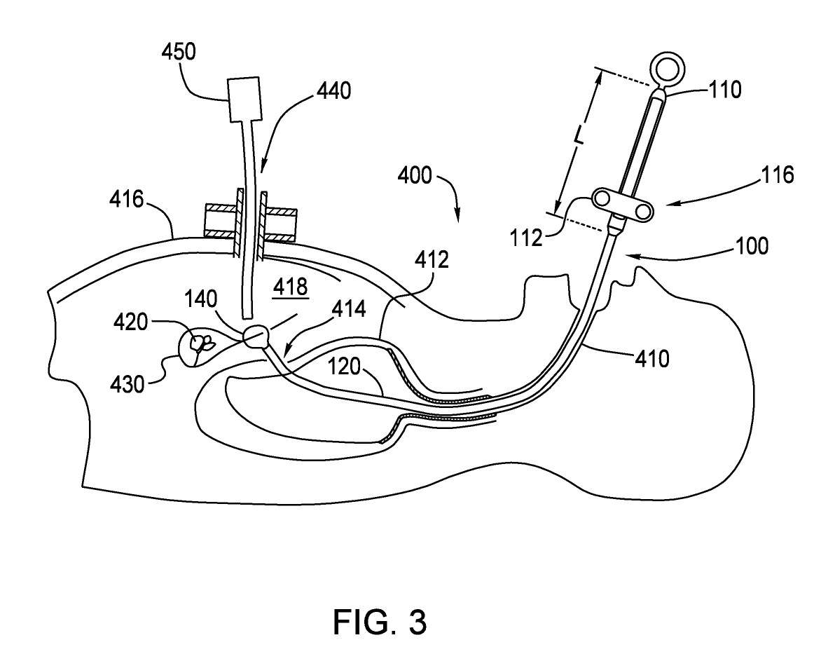

The surgical device 100 may be utilized to extract material located internally within a patient. As described above, the material extracted from the patient may be an organ, tissue, or a foreign object. As shown in FIG. 3, in one embodiment to extract material from a patient 400, the surgical device 100 is inserted into an esophagus 410 of the patient 400 in need of such extraction. Insertion of the surgical device 100 is done using generally known procedures while the patient 400 is sedated. The surgical device 100 extends through the esophagus 410 to a stomach 412 having an incision 414 therein. The incision 414 may be made by the surgical device 100. Alternatively, the incision 414 may be made by another surgical device inserted through an incision made in the torso 416 of the patient 400.

After insertion into the esophagus, the surgical device 100 is passed through the incision 414 to an internal cavity 418 of the patient 400 such that the surgical device 100 is proximate to material 420 in need of extraction.

In one embodiment, at least a portion of the material 420 in need of extraction is surrounded by an extraction pouch 430. Extraction pouch 430 may be any device that is capable of surrounding at least a portion of the material 420 in need of extraction, and includes, but is not limited to a bag, a net, a loop, or the like. Extraction pouch 430 may be introduced to the patient 400 through an incision 440. In one example the device 450 is a camera inserted through the incision 440 to aid the surgeon in performing the procedure.

As shown in FIG. 3, when the actuator 112 of the surgical device 100 is in the second position 116 along the length L of the handle 110 at least a portion of the surgical instrument 140 extends from the second end 124 of the first flexible conduit 120. While not shown in FIG. 3, it is contemplated that surgical device 100 may also include a second flexible conduit having a second end which at least a portion of the surgical instrument 140 extends through. Additionally, the surgical device 100 may also include an anti-reflux valve coupled to the second flexible conduit to provide a gas to the stomach 412 of the patient 400.

In one embodiment, the surgical device 100 is positioned in a manner that allows the surgical instrument 140 to grasp or otherwise loop around the material 420 in need of extraction. As shown in FIG. 3, the surgical instrument 140 is a resection loop that has been positioned around at least a portion of the material 420 in need of extraction. The invention is not limited in this regard as the surgical instrument 140 may be another device, such as, for example, clamps, tweezers, a resection device such as scissors, and articulating arms, and the like, which are capable of grasping or holding the material 420.

After positioning the surgical instrument 140 and the extraction pouch 430 in the desired locations within the patient 400, the actuator 112 is moved to the first position 114 along the length L of the handle 110, thereby actuating the surgical instrument 140 to grippingly retain at least a portion of the extraction pouch 430. The manner in which the surgical instrument 140 grippingly retains at least a portion of the extraction pouch 430 is dependent upon what type of surgical instrument is employed with the surgical device 100. For example, if the surgical instrument 140 is a resection loop, when the actuator 112 is moved to the first position 114 along the length L of the handle 110, the resection loop is pulled back within the first flexible conduit 120, thereby decreasing the size of the loop that is positioned around at least a portion of the extraction pouch 430. In another example, when the surgical instrument 140 is a clamp, the clamp is in a closed position to grippingly retain at least a portion of the extraction pouch 430.

Once at least a portion of the extraction pouch 430 is grippingly retained by the surgical instrument 140, the surgical device 100 is withdrawn from the patient, thereby removing the extraction pouch and the material 420 from the patient 400. Removal or extraction of the material 420 from the patient 400 via the esophagus 410 allows a surgeon to remove material that may otherwise not be capable of removal by utilizing traditional laparoscopic surgical methods.

Although the present invention has been disclosed and described with reference to certain embodiments thereof, it should be noted that other variations and modifications may be made, and it is intended that the following claims cover the variations and modifications within the true scope of the invention.

What is claimed is:

1. A surgical device for extracting material located inside a patient, the surgical device comprising:
a handle;
an actuator slidably disposed in the handle and movable relative thereto between a first position and a second position;
a first flexible conduit having a first end coupled to the handle, the first flexible conduit defining a bore extending therethrough;
a flexible actuation line having a first end coupled to the actuator, the actuation line extending through the bore defined by the first flexible conduit; and
a surgical instrument coupled to a second end of the actuation line;
wherein upon movement of the actuator from the first position to the second position the surgical instrument moves from a retracted position within the bore defined by the first flexible conduit to an extended position wherein the surgical instrument extends at least partially from a second end of the first flexible conduit;
wherein the surgical instrument is actuated to grippingly retain material inside a patient upon movement of the actuator from the second position to the first position.

2. A surgical device according to claim 1, wherein the surgical instrument is selected from a group consisting of a resection loop, clamps, pincers, a blade, a tweezer, articulating arms, and a combination thereof.

3. A surgical device according to claim 2, wherein the surgical instrument is a resection loop.

4. A method for extracting material inside a patient, the method comprising the steps of:
providing a surgical device according to claim 1;
inserting the second end of the first flexible conduit into an esophagus of a patient;
extending the second end of the first flexible conduit through the esophagus to a stomach having an incision therein;
extending the second end of the first flexible conduit through the incision in the stomach to an internal cavity of the patient such that the second end of the first flexible conduit is proximate to a material in need of extraction;
surrounding a least a portion of the material with an extraction pouch;
moving the actuator of the surgical device to the second position thereby extending at least a portion of the surgical instrument from the second end of the first flexible conduit;
moving the actuator to the first position, thereby actuating the surgical instrument to grippingly retain at least a portion of the extraction pouch; and
withdrawing the surgical instrument from the patient, thereby removing the extraction pouch and material from the patient.

5. A method according to claim 4, wherein the surgical instrument is selected from a group consisting of a resection loop, clamps, pincers, a blade, a tweezer, an articulating arm, and a combination thereof.

6. A method according to claim 5, wherein the surgical instrument is a resection loop.

7. A method according to claim 5, further comprising the step of:
disposing the first flexible conduit of the surgical device into a second flexible conduit defining a bore, wherein at least a portion of the first flexible conduit extends through the bore of the second flexible conduit such that the first end of the first flexible conduit is aligned with a first end of the second flexible conduit.
